(19) United States
(12) Patent Application Publication
(10) Pub. No.: US 2011/0038928 A1
(43) Pub. Date: Feb. 17, 2011

(54) ORALLY DISINTEGRATING TABLETS OF ZOLMITRIPTAN

(75) Inventors: Kamal Mehta, Bhilwara (IN); Srinivas Ganga Arra, Navi Mumbai (IN); Chandramala A. Kaushal, Navi Mumbai (IN); Manish Maheshwari, Navi Mumbai (IN)

Correspondence Address: GLENMARK GENERICS INC., 750 CORPORATE DRIVE, MAHWAH, NJ, 07430, US

(73) Assignee: GLENMARK GENERICS LTD, Mumbai (IN)

(21) Appl. No.: 12/853,500

(22) Filed: Aug. 10, 2010

(30) Foreign Application Priority Data
Aug. 12, 2009 (IN) 1862/MUM/2009

(51) Int. Cl. A61K 9/20; A61K 31/422; A61P 25/06

(52) U.S. Cl. 424/465; 514/376; 424/464

(57) ABSTRACT

The present invention is directed to a novel rapidly dissolvable pharmaceutical composition comprising zolmitriptan or pharmaceutically acceptable salts or esters thereof and least one direct compression filler. The pharmaceutical compositions of the present invention can advantageously be prepared in a solid dosage form, e.g. a tablet, utilizing directly compressible ingredients.

### PRIORITY

This application claims priority to Indian Provisional Application No. 1862/MUM/2009, filed on Aug. 12, 2009, the contents of which, is hereby incorporated by reference.

### BACKGROUND OF THE INVENTION

1. Technical Field

The present invention generally relates to an orally disintegrating tablet comprising zolmitriptan or pharmaceutically acceptable salts or esters thereof and to a process for preparing the same.

2. Description of Related Art

Zolmitriptan, chemically known as S)-4-[[3-[2-(dimethylamino) ethyl]-1H-indol-5-yl]methyl]-2-oxazolidinone. is represented below as I.

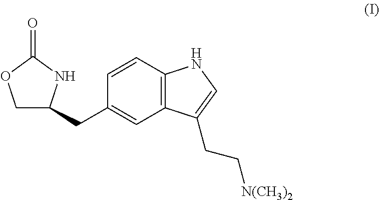

Zolmitriptan is a selective 5-hydroxy-tryptamine1B/1D (5-HT1B/1D) receptor agonist. Zolmitriptan and its use in the treatment and prophylaxis of migraine was first disclosed in U.S. Pat. No. 5,466,699. Zolmitriptan is marketed in the US as ZOMIG®, which is a film coated tablet and as ZOMIG-ZMT®, which is an orally disintegrating tablet.

ZOMIG-ZMT® tablets are available as 2.5 mg and 5.0 mg white uncoated tablets for oral administration and these tablets contain mannitol USP, microcrystalline cellulose NF, crospovidone NF, aspartame NF, sodium bicarbonate USP, citric acid anhydrous USP, colloidal silicon dioxide NF, magnesium stearate NF and orange flavor SN 027512.

### SUMMARY OF THE INVENTION

The present invention provides a novel rapidly dissolvable pharmaceutical composition comprising zolmitriptan and pharmaceutically acceptable salts or esters thereof.

In one of the aspect, the present invention provides, a novel rapidly dissolvable pharmaceutical composition comprising zolmitriptan or pharmaceutically acceptable salts or esters thereof and at least one direct compression filler.

In another aspect, the present invention provides a novel rapidly dissolvable pharmaceutical composition comprising zolmitriptan or pharmaceutically acceptable salts or esters thereof, at least one direct compression filler & disintegrating agent.

In one more aspect, the present invention provides a novel rapidly dissolvable pharmaceutical composition comprising zolmitriptan or pharmaceutically acceptable salts or esters thereof, at least one direct compression filler, disintegrating agent & lubricant.

In another aspect, the present invention provides a novel rapidly dissolvable pharmaceutical composition comprising zolmitriptan or pharmaceutically acceptable salts or esters thereof and at least one direct compression filler; wherein said filler is in spray dried, granulated, compacted or agglomerated form.

The present invention provides a novel rapidly dissolvable pharmaceutical composition comprising zolmitriptan or pharmaceutically acceptable salts or esters thereof and at least one direct compression filler, wherein the filler has a mean diameter of from about 60 microns to about 500 microns, preferably from about 80 microns to about 400 microns, and more preferably from about 150 microns to about 300 microns.

In another aspect, the present invention provides a process for the preparation of a novel rapidly dissolvable pharmaceutical composition comprising zolmitriptan or pharmaceutically acceptable salts or esters thereof and at least one direct compression filler, wherein said filler is in spray dried, granulated, compacted or agglomerated form comprising: (a) sifting a zolmitriptan, or pharmaceutically acceptable salts or esters thereof, and direct compression filler, then (b) uniformly mixing of sifted materials; and (d) compressing the blend into tablets.

In one more aspect, the present invention provides a process for the preparation of a novel rapidly dissolvable pharmaceutical composition comprising zolmitriptan or pharmaceutically acceptable salts or esters thereof and at least one direct compression filler, wherein said filler is in spray dried, granulated, compacted or agglomerated form comprising: (a) granulating/agglomerating a non-direct compression filler optionally with binder and/or disintegrant and sizing said filler to produce particles of mean diameter of from about 60 microns to about 500 microns, and preferably from about 80 microns to about 400 microns, and more preferably from about 150 microns to about 300 microns; (b) mixing granules of (a) with sifted zolmitriptan and lubricant and then (c) compression into tablets.

### DETAILED DESCRIPTION OF THE PREFERRED EMBODIMENTS

The present invention is directed to a novel rapidly dissolvable pharmaceutical composition comprising zolmitriptan or pharmaceutically acceptable salts or esters thereof and at least one direct compression filler, and a process for the preparation thereof. The pharmaceutical compositions of the present invention can advantageously be prepared in a solid dosage form, e.g. a tablet, utilizing directly compressible ingredients.

There are several pharmaceutical techniques to make the orally disintegrating tablets, however few of such techniques use effervescent agent for fast disintegration of tablets.

U.S. Pat. No. 5,178,878, included herein as reference in its entirety, discloses a solid tablet dosage form comprising a mixture of at least one saliva activated effervescent agent and a plurality of microparticles, wherein each microparticle includes at least one systemically distributable active principle and a protective material substantially encompassing the active principle.

U.S. Pat. No. 5,464,632 (“the '632 patent”) discloses a rapidly disintegratable tablet comprising an active substance and a mixture of excipients, wherein the active substance is multiparticulate and in the form of coated microcrystals, coated microgranules or uncoated microgranules. The '632 patent describes an active ingredient as either coated or granulated using a complex manufacturing technique.

U.S. Pat. No. 6,024,981 and U.S. Pat. No. 6,221,392 (“the '981 patent” and “the '392 patent”) disclose hard, compressed, rapidly dissolvable tablets comprising an active ingredient and a matrix including a non-direct compression filler and a lubricant, having a friability of about 2% or less and hardness of at least 15 Newtons. The non-direct compression filler of the '981 & '392 patents have average particle sizes that range from between about 10 and about 80 microns, and most preferably, between about 20 to about 65 microns.

Prior art described herein above, involves the techniques like effervescence agent, non-direct vehicle, coating or an encompassing active principle to make oral disintegrating tablets. Disadvantageously, the prior art techniques are largely complex thus are both difficult to practice and can be time consuming. Therefore, techniques to make oral disintegrating or fast dissolving tablets, which are less complex and facile, are highly desired.

The tablet of the present invention can include a therapeutically effective amount of daily dose of zolmitriptan and pharmaceutically acceptable salts or esters thereof, from about 0.5 mg to about 100.0 mg; preferably from about 1.25 mg to about 50.0 mg, more preferably from about 2.5 mg to about 50.0 mg.

The present invention provides a novel rapidly dissolvable pharmaceutical composition comprising zolmitriptan and pharmaceutically acceptable salts or esters thereof, wherein the zolmitriptan and pharmaceutically acceptable salts or esters thereof range from about 2.0 wt % to about 20.0 wt %; preferably from about 2.5 wt % to about 10.0 wt % and a direct compressible filler ranging from about 20.0 wt % to about 98.0 wt %; preferably from about 40 wt % to 90.0 wt %, all relative to total tablet weight.

The present invention provides a novel rapidly dissolvable pharmaceutical composition comprising zolmitriptan and pharmaceutically acceptable salts or esters thereof, wherein the zolmitriptan and pharmaceutically acceptable salts or esters thereof range from about 2.0 wt % to about 20.0 wt %; preferably from about 2.5 wt % to about 10.0 wt %, and a direct compressible filler ranging from about 20.0 wt % to about 98.0 wt %; more preferably from about 40 wt % to 90.0 wt % and a disintegrating agent ranging from about 1.0 wt % to about 40.0 wt %; and more preferably from about 2.0 wt % to 20.0 wt %, all based on total tablet weight.

A novel rapidly dissolvable pharmaceutical composition comprising zolmitriptan and pharmaceutically acceptable salts or esters thereof, wherein the zolmitriptan and pharmaceutically acceptable salts or esters thereof range from about 2.0 wt % to about 20.0 wt %; preferably from about 2.5 wt % to about 10.0 wt %, and a direct compressible filler ranging from about 20.0 wt % to about 98.0 wt %; preferably from about 40 wt % to 90.0 wt %, and a disintegrating agent ranging from about 1.0 wt % to about 40.0 wt %; preferably from about 2.0 wt % to 20.0 wt % and a lubricant ranging from 0.5 wt % to 2.5 wt %; preferably from 0.5 wt % to 1.5 wt % or less than 1.0 wt %, all weights relative to total tablet weight.

A novel rapidly dissolvable pharmaceutical composition comprising zolmitriptan and pharmaceutically acceptable salts or esters thereof, wherein the zolmitriptan and pharmaceutically acceptable salts or esters thereof range from about 2.0 wt % to about 20.0 wt %; preferably from about 2.5 wt % to about 10.0 wt %, and a direct compressible filler from about 20.0 wt % to about 98.0 wt %; preferably from about 40 wt % to 90.0 wt %, and a disintegrating agent from about 1.0 wt % to about 40.0 wt %; preferably from about 2.0 wt % to 20.0 wt % and a lubricant ranging from 0.5 wt % to 2.5 wt %; preferably from 0.5 wt % to 1.5 wt % or less than 1.0 wt %, all relative to total tablet weight. Further, this dosage form may further comprise diluents or bulking agent ranging from 10.0 to 80.0 wt %; preferably 20.0 to 50.0 wt %, and a sweetening agent ranging from about 1.0 wt % to about 20 wt %; preferably from about 2.0 wt % to about 10 wt % and a flavoring agent ranging from about 0.05 wt % to about 2.0 wt %; preferably from about 0.1 wt % to about 1.5 wt %, all relative to total tablet weight.

The present invention provides a novel rapidly dissolvable pharmaceutical composition adapted for direct oral dosing in a solid dosage form may additionally comprise glidants, antioxidants, colorants, wetting agents, buffering agents and an effervescent agent.

The phrase “rapidly dissolvable” as used herein refers, to the ability of a tablet to disintegrate rapidly when contacted with a fluid, particularly water or saliva, to form a suspension or slurry which can be easily swallowed.

The phrase “treating or treatment of a state, disorder or condition” as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The phrase “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The phrase “pharmaceutically acceptable salts and esters” as used herein refers to those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, lauryl sulphate salts and the like. Representative alkali or alkaline earth metal salts include the sodium, calcium, potassium and magnesium salts, and the like.

The present invention uses excipients selected from the category of “directly compressible” fillers, as used herein, which are in granulated, compacted or agglomerated form; such as, directly compressible mannitol, directly compressible sorbitol, directly compressible maltitol, directly compressible lactose, directly compressible sucrose, directly compressible xylose, directly compressible trehalose, directly compressible dextrose, directly compressible lactose, directly compressible microcrystalline cellulose and the like, and combinations thereof. Preferably a directly compressible mannitol can be selected from the commercially available grades of direct compressible mannitol such as PEARLITOL® 200 SD, PEARLITOL® 300 DC, PEARLITOL® 400 DC, PEARLITOL® 500 DC (manufactured by Roquette Lestrem, France), Parteck™ M 200 and Parteck™ M 300 (manufactured by Merck KGaA, Darmstadt, Germany).

The phrase, “non-direct compressible fillers”, as used herein, refers to physically unmodified powdered or fine grained materials such as mannitol, sorbitol, maltitol, lactose, sucrose, xylose, dextrose, lactose, microcrystalline cellulose etc.

The phrase “sweetening agent”, as used herein, is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art. Preferably, aspartame.

The term “binders”, as used herein, is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab™), ethylcellulose, gelatin, liquid glucose, methylcellulose, povidone and pregelatinized starch, combinations thereof and other material known to those of ordinary skill in the art. Preferably, poly(vinylpyrrolidone).

The term “diluent” or “filler” or phrase “bulking agent” as used herein, is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art. Preferably, microcrystalline cellulose.

The term “glidant”, as used herein, is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art. Preferably, colloidal silica or talc.

The term “lubricant”, as used herein, is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art. Preferably, magnesium stearate.

The term “disintegrant”, as used herein, is intended to mean a compound used in solid dosage forms to promote the disruption of the solid mass into smaller particles which are more readily dispersed or dissolved. Exemplary disintegrants include, by way of example and without limitation, crospovidone (Polyplasdone® XL10), croscarmellose (ac di sol), starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art. Preferably, crospovidone (Polyplasdone® XL10).

The term “wetting agent”, as used herein, is intended to mean a compound used to aid in attaining intimate contact between solid particles and liquids. Exemplary wetting agents include, by way of example and without limitation, gelatin, casein, lecithin (phosphatides), gum acacia, cholesterol, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearate, sodium dodecylsulfate, combinations thereof and other such materials known to those of ordinary skill in the art. Preferably, sorbitan esters.

As one skilled in the art will readily appreciate, the excipients of the present invention may function as more than one type of excipient, e.g. mannitol may act as a diluent and a sweetening agent.

Most of these excipients are described in detail in Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

The present invention may apply to other agents, illustratively, neuroleptics and psychopharmacological agents, e.g., antipsychotic drugs and antidepressant drugs. Some common psychopharmacological agents are described in Remington, The Science and Practice of Pharmacy 20th Ed. and is incorporated herein by reference. Psychotropic agents may include anti-anxiety, antidepressant, anti-manic, anti-panic, antipsychotic, anti-migraine, or phenothiazines, or combinations thereof. Representative examples of antipsychotic drugs for use may include fluphenazine, decanoate, haloperidol, loxapine, thiothixene, clozapine, olanzapine, risperidone and the like. Representative examples of antidepressant drugs for use herein may include amoxapine, fluvoxamine, imipramine pamoate, mirtazapine, trazodone, trimipramine and the like. Examples of neuroleptics for use herein may include decarboxylase inhibitors such as carbidopa and levodopa as well as catechol methyltransferase inhibitors such as entacapone. Representative examples of anti-migraine drugs are zolmitriptan, rizatriptan, samaritan and others, or their pharmaceutically acceptable salts, isomers, metabolites, polymorphic forms, etc.

The composition of the present invention may optionally include, but is not limited to, buffering agent, for example, sodium hydrogen phosphate, sodium hydrogen biphosphate. The preferred buffering agent of the present invention is sodium hydrogen biphosphate.

Suitable colorants can include, for example, red, black and yellow iron oxides; FD & C dyes (e.g., FD & C blue No. 2, FD & C red No. 40); and the like, and combinations thereof.

Suitable flavorants can include, for example, flavors, which are known to those of ordinary skill in the art, such as, natural flavors, artificial flavors, and combinations thereof. Flavorants may be chosen, e.g., from synthetic flavor oils and flavoring aromatics and/or oils, oleoresins, extracts derived from plants, leaves, flowers, fruits, and the like, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic fruit flavors such as vanilla, citrus oils (e.g., lemon, orange, lime, and grapefruit), and fruit essences (e.g., apple, pear, peach, grape, strawberry, raspberry, cherry, plum, pineapple, and apricot), and the like, and combinations thereof.

The phrase “effervescent agent”, as used herein, includes compounds which evolve gas. The preferred effervescent agents evolve gas by means of a chemical reaction which takes place upon exposure of the effervescent agent (an effervescent couple) to water and/or to saliva in the mouth. This reaction is most often the result of the reaction of a soluble acid source and a source of carbon dioxide such as an alkaline carbonate or bicarbonate. The acid sources may be any which are safe for human consumption and may generally include food acids, acid and hydrate antacids such as, for example: citric, tartaric, amalic, fumeric, adipic, and succinics. Carbonate sources include dry solid carbonate and bicarbonate salt such as, preferably, sodium bicarbonate, sodium carbonate, potassium bicarbonate and potassium carbonate, magnesium carbonate and the like. Reactants which evolve oxygen or other gases and which are safe for human consumption are also included.

Inert excipients selected for making the tablets of the present invention are prepared in such a way that they are directly compressible in nature. Also, the excipients of the present invention may have relatively low moisture content.

The following examples are provided to enable one skilled in the art to practice the invention and are merely illustrative of the invention. The examples should not be read as limiting the scope of the invention as defined in the claims.

### EXAMPLES

### Example 1

### Preparation of Zolmitriptan Orally Disintegrating Tablet by Direct Compression

The ingredients and amounts for this example are set forth below in Table 1.

TABLE 1

| Sr. No. | Ingredients | Mg/Tab |
|---|---|---|
| 1 | Zolmitriptan | 5.00 |
| 2 | Mannitol (Pearlitol ® SD 200) | 86.50 |
| 3 | Microcrystalline cellulose (MCC) | 88.90 |
| | (Avicel ® PH 112) | |
| 4 | Crospovidone (Polyplasdone ® XL10) | 6.00 |
| 5 | Aspartame | 10.00 |
| 6 | Orange Flavour (PB82 #739K) | 0.60 |
| 8 | Mg Stearate | 3.00 |
| | Tablet Weight | 200.0 |

Brief Manufacturing Procedure
1. Zolmitriptan, Mannitol, Crospovidone, aspartame, Orange flavour and MCC were sifted through #40 and loaded into a blender and mixed for about 5 minutes, then Mg-Stearate (Sifted through #40) was added and mixed for about 3 minutes,
2. The blend from no. 1 was then compressed to tablets.

### Example 2

### Preparation of Zolmitriptan Orally Disintegrating Tablets by Dry Granulation

The ingredients and amounts for this example are set forth below in Table 2.

TABLE 2

| Sr. | | |
|---|---|---|
| No. | Ingredient | Mg/tab |
| 1. | Zolmitriptan | 5.00 |
| 2. | Mannitol (Pearlitol ® 300 DC) | 124.00 |
| 3. | Microcrystalline Cellulose (Avicel ® PH | 52.4 |
| | 112) | |
| 4. | Crospovidone (Polyplasdone ® XL 10) | 10.00 |
| 5. | Aspartame (Nutrasweet ®) | 4.00 |
| 6. | Orange Flavor PB82#739K | 0.60 |
| 7. | Colloidal silicon dioxide (Aerosil ® 200) | 1.0 |
| 8. | Mg Stearate | 3.00 |
| | Total Weight (mg) | 200.0 |

Brief Manufacturing Procedure
1. Zolmitriptan, Mannitol, MCC, and part of Crospovidone were sifted through #30 sieve and loaded into Bin Blender and mixed for about 5 minutes. Part of Mg-stearate Sifted through 60# sieve was added and mixed for about 2 minutes.
2. The blend of 1 was then roll compacted and milled through 1.5 mm screen and then mixed with aspartame, the remaining part of Crospovidone, orange flavors, colloidal silicon dioxide Sifted (through # 40 sieve) and added Mg-stearate.
3. The blend of 2 was then compressed to tablets.

### Example 3

### Preparation of Zolmitriptan Orally Disintegrating Tablets by Wet Granulation

The ingredients and amounts for this example are set forth below in Table 3.

TABLE 3

| Sr. | | |
|---|---|---|
| No. | Ingredient | Mg/tab |
| 1. | Mannitol (Pearlitol 300 DC) | 124.00 |
| 2. | Microcrystalline Cellulose (Avicel PH 112) | 47.4 |
| 3. | Crospovidone (Polyplasdone XL 10) | 5.00 |
| 4. | Purified Water | qs |
| 5. | Zolmitriptan | 5.00 |
| 6. | Crospovidone (Polyplasdone XL 10) | 10.0 |
| 7. | Aspartame (NutraSweet) | 4.00 |
| 8. | Orange Flavor PB82#739K | 0.60 |
| 9. | Colloidal silicon dioxide (Aerosil 200) | 1.00 |
| 10. | Mg Stearate | 3.00 |

| | |
|---|---|
| Total Weight (mg) | 200.0 |

Brief Manufacturing Procedure
1. Co-sifted (# 30 sieve) Mannitol, MCC and Crospovidone were added into rapid mixer Granulator (RMG) and mixed for about 5 minutes. Said mixer was then granulated using purified water.
2. The wet mass of 1 was dried at about 60° C. and passed through #30.
3. Fines of 2 mixed with Zolmitriptan and passed through sieve # 60.
4. Granules from 2 along with drug-fines mixer of 3 loaded into blender and mixed for about 2 minutes.
5. Sifted (through # 40) Crospovidone, Aspartame, Orange Flavour, and Colloidal silicon dioxide were added to 4 and blended for about 5 minutes.
6. Then Mg stearate sifted through sieve #60 was added to 5 and blended for about 3 minutes.
7. The blend of 6 was then compressed to tablets.

### Example 4

### Preparation of Zolmitriptan Orally Disintegrating Tablets Comprising Effervescent Agent, by Dry Granulation

The ingredients and amounts for this example are set forth below in Table 4

TABLE 4

| Sr. | | |
|---|---|---|
| No. | Ingredient | Mg/tab |
| 1. | Zolmitriptan | 5.00 |
| 2. | Mannitol (Pearlitol ® 300 DC) | 124.00 |
| 3. | Microcrystalline Cellulose (Avicel ® PH | 48.1 |
| | 112) | |
| 4. | Crospovidone (Polyplasdone ® XL 10) | 10.00 |
| 5. | Aspartame (Nutrasweet ®) | 4.00 |
| 6. | Orange Flavor PB82#739K | 0.60 |
| 7. | Colloidal silicon dioxide (Aerosil ® 200) | 1.0 |
| 8. | Citric Acid Anhydrous (Emprove ®) | 1.8 |
| 9. | Sodium Bicarbonate (Emprove ®) | 2.5 |
| 10. | Mg Stearate | 3.00 |

| | |
|---|---|
| Weight (mg) | 200.0 |

Brief Manufacturing Procedure
1. Co-sifted (through #30 sieve) Zolmitriptan, Mannitol, MCC, Citric Acid Anhydrous,
Sodium Bicarbonate and part of Crospovidone were loaded into Bin Blender and mixed for about 5 minutes. Part of Mg-stearate sifted through sieve #60 was then added to the above blend and mixed for about 2 minutes.
2. The blend of 1 was then roll compacted and milled through 1.5 mm screen and then mixed with aspartame, remaining part of Crospovidone, orange flavors, pre-sifted colloidal silicon dioxide (through # 40 sieve) and Mg-stearate.
3. The blend of 2 was then compressed to tablets.

### Example 5

### Preparation of Zolmitriptan Orally Disintegrating Table Comprising Effervescent Agent, by Direct Compression

The ingredients and amounts for this example are set forth below in Table 5.

TABLE 5

| Sr. | | |
|---|---|---|
| No. | Ingredient | Mg/tab |
| 1. | Zolmitriptan | 5.00 |
| 2. | Mannitol (Pearlitol ® 300 DC) | 124.00 |
| 3. | Microcrystalline Cellulose (Avicel ® PH | 48.1 |
| | 112) | |
| 4. | Crospovidone (Polyplasdone ® XL 10) | 10.00 |
| 5. | Aspartame (Nutrasweet) | 4.00 |
| 6. | Orange Flavor PB82#739K | 0.60 |
| 7. | Colloidal silicon dioxide (Aerosil ® 200) | 1.0 |
| 8. | Citric Acid Anhydrous (Emprove ®) | 1.8 |
| 9. | Sodium Bicarbonate (Emprove ®) | 2.5 |
| 10. | Mg Stearate | 3.00 |

| | |
|---|---|
| Weight (mg) | 200.0 |

Brief Manufacturing Procedure
1. Co-sifted (through #30 sieve) Zolmitriptan, Mannitol, aspartame, Orange flavour, MCC, Citric Acid Anhydrous, Sodium Bicarbonate, colloidal silicon dioxide and Crospovidone were loaded into Bin Blender and mixed for 5 minutes, then Mg-Stearate (Sifted through sieve #40) was added and mixed for about 3 minutes.
2. The blend from 1 was then compressed to tablets.

### Example 6

### Preparation of Zolmitriptan Orally Disintegrating Table Comprising Effervescent Agent, by Wet Granulation

The ingredients and amounts for this example are set forth below in Table 6.

TABLE 6

| Sr. No. | Ingredient | Mg/tab |
|---|---|---|
| 1. | Mannitol (Pearlitol 300 DC) | 124.00 |
| 2. | Microcrystalline Cellulose (Avicel ® PH 112) | 43.1 |
| 3. | Crospovidone (Polyplasdone ® XL 10) | 5.00 |
| 4. | Purified Water | |
| 5. | Zolmitriptan | 5.00 |
| 6. | Crospovidone (Polyplasdone ® XL 10) | 10.0 |
| 7. | Aspartame (NutraSweet ®) | 4.00 |
| 8. | Orange Flavor PB82#739K | 0.60 |
| 9. | Citric Acid Anhydrous (Emprove ®) | 1.8 |
| 10. | Sodium Bicarbonate (Emprove ®) | 2.5 |
| 11. | Colloidal silicon dioxide (Aerosil ® 200) | 1.0 |
| 12. | Mg Stearate | 23.00 |

| | |
|---|---|
| Weight (mg) | 200.0 |

Brief Manufacturing Procedure
1. Co-sifted (# 30 sieve) Mannitol, MCC and Crospovidone were added into rapid mixer granulator and mixed for about 5 minutes. Said mixer was then granulated using purified water.
2. The wet mass of 1 then dried at about 60° C. and passed through sieve #30.
3. Fines of 2 were mixed with Zolmitriptan and passed through sieve # 60
4. Granules from 2 along with drug-fines mixer of 3 loaded into blender and mixed for about 2 minutes.
5. Sifted (through sieve # 40) Crospovidone, Aspartame, Orange Flavour, Citric acid anhydrous, Sodium bicarbonate and Colloidal silicon dioxide were added to 4 and blended for about 5 minutes.
6. Then Mg stearate sifted through sieve #60 was added to 5 and blended for about 3 minutes.
7. The blend of 6 was then compressed to tablets.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention.

1. A rapidly dissolvable pharmaceutical composition comprising zolmitriptan or pharmaceutically acceptable salts or esters thereof and at least one direct compression filler, wherein said filler is in spray dried, granulated, compacted or in agglomerated form.

2. The composition of claim 1, wherein the direct compression filler is selected from the group comprising directly compressible mannitol, directly compressible sorbitol, directly compressible maltitol, directly compressible lactose, directly compressible sucrose, directly compressible xylose, directly compressible trehalose, directly compressible dextrose, directly compressible lactose, directly compressible microcrystalline cellulose.

3. The composition of claim 2, wherein the direct compression filler ranges from about 20.0 wt % to about 98.0 wt %.

4. The composition of claim 3, wherein the direct compression filler has a mean diameter of from about 60 microns to about 500 microns.

5. The composition of claim 1, further comprises a disintegrating agent and a lubricant.

6. The composition of claim 5, wherein the disintegrating agent ranges from about 1.0 wt % to about 40.0 wt %, and wherein the lubricant ranges from 0.5 wt % to 2.5 wt %.

7. A process for the preparation of rapidly dissolvable pharmaceutical composition comprising zolmitriptan or pharmaceutically acceptable salts or esters thereof and at least one direct compression filler, wherein said filler is in spray dried, granulated, compacted or in agglomerated form comprising: (a) sifting a zolmitriptan, or pharmaceutically acceptable salts or esters thereof, and a direct compression filler, then (b) uniformly mixing of sifted materials; and (c) compressing the blend into tablets.

8. A process for the preparation of a novel rapidly dissolvable pharmaceutical composition comprising zolmitriptan or pharmaceutically acceptable salts or esters thereof and at least one direct compression filler, wherein said filler is in spray dried, granulated, compacted or in agglomerated form, comprising: (a) granulating/agglomerating a non-direct compression filler optionally with binder and/or disintegrant and sizing said filler, to produce particles of mean diameter of from about 60 microns to about 500 microns; (b) mixing granules of (a) with pre-sifted zolmitriptan and lubricant and then (c) compression into tablets.

9. The process of claim 8, wherein the particles of (a) are from about 80 microns to 400 microns.

10. The process of claim 9, wherein the particles of (a) are from about 150 microns to about 300 microns.
